(19) United States
(12) Patent Application Publication
(10) Pub. No.: US 2010/0003223 A1
(43) Pub. Date: Jan. 7, 2010

(54) METHOD FOR THE PRODUCTION OF MULTICOMPONENT STEM CELLS, RELATIVE KITS AND USES IN THE MEDICAL FIELD

(75) Inventors: Rosa Anna Cifarelli, Metaponto (Matera) (IT); Francesco Cellini, Metaponto (Matera) (IT); Rosa Di Liddo, Padova (IT); Pier Paolo Parnigotto, Padova (IT)

Correspondence Address: HEDMAN & COSTIGAN P.C., 1185 AVENUE OF THE AMERICAS, NEW YORK, NY, 10036, US

(73) Assignee: Metapontium Agrobios S.R.L., Metaponto (IT)

(21) Appl. No.: 12/310,236

(22) PCT Filed: Jul. 26, 2007
(86) PCT No.: PCT/IB2007/002204

§ 371 Date: Aug. 28, 2009

(30) Foreign Application Priority Data
Jul. 28, 2006 (IT) MI2006A001498

(51) Int. Cl. A61K 35/12; C12N 5/08; A61P 43/00

(52) U.S. Cl. 424/93.7; 435/366

(57) ABSTRACT

The invention relates to a method for the production of multipotent stem cells starting from highly differentiated adult somatic cells of mammals or their precursors comprising the demethylating treatment phase of highly differentiated cells with 5′ Aza 2′ cytidine and relative kits and uses in the medical field.

The present invention relates to a new method for the production of multipotent stem cells, the relative kits and uses in the medical field.

Research on multipotent and totipotent stem cells (Conrad C., et al., 2005) has opened up new and interesting applicative prospects for cellular therapy and tissue engineering. What the most suitable stem cell source is for applicative purposes, however, is still to be defined.

The use of embryonic stem populations (ES) has recently stimulated a growing scientific interest in the medical field. Although these cells are characterized by a high replication capacity, differentiative pluripotentiality and long-term expansion in vitro, their therapeutic use, however, is limited due to the numerous ethical problems involved (Kiatpongsan S. et al., 2006).

Considerable experimental evidence has demonstrated that alternative sources of stem cells can be identified in adults; although these only have the capacity of differentiating in a limited number of cellular types, they do not arouse ethical problems. Among the sources of adult stem cells, bone marrow (Saulnier N. et al., 2005), seminiferous epithelium of the male gonad (Cyranoski D. et al., 2006), epithelia (Janes S M, et al., 2002), peripheral blood, cord blood (Saulnier N. et al., 2005) and adipose tissue (Strem B M, et al., 2005).

Adult stem cells, however, are not easily available as they are numerically limited; furthermore they cannot be cultivated in vitro for a long period as, after various cell divisions, they tend to lose their multipotentiality.

Together with this physiological stem cell sources, another extremely promising source has recently been added, which can be obtained by modification of the differentiated cell genetic program (F. Santos, et al., 2002; A. J. Peter, et al., 2001; R. Wolf, et al., 2001; J. C. Gutierrez, et al., 2000; T. H. Bestor, 2000; C. Stewart, et al., 1982; R. D. Palmiter, et al., 1982; D. Biniszkiewicz, et al., 2002; M. F. Chan, et al., 2001; M. Okano, et al., 1999). The researchers Wilmut and Campbell (Shiels P G, et al., 1999; Eyestone W H, et al., 1999) and subsequently Yanagimachi and collaborators (Wakayama T, et al., 2001) clearly established, using animal models, that the nucleus of completely differentiated somatic cells can be reprogrammed if transferred into the cytoplasm of an egg cell. The mechanisms and molecules involved in this deprogramming and reprogramming process of the genome of somatic cells has not yet been completely clarified. It is clear, however, that during this process the chromatin is decondensed, the nucleosome structure is destabilized and the regulator proteins are dissociated from the DNA fibre thus influencing the gene expression (T. Haaf, et al., 2000).

Other experimental observations have shown that, in the first embryonic development phases, there are numerous epigenetic methylation modifications of the cytosine residues with respect to the genome, as a result of which there is a change in the cell phenotype and commitment of the cell towards a specific differentiative line (F. Santos, et al., 2002; A. J. Peter, et al., 2001; R. Wolf, et al., 2001; J. C. Gutierrez, et al., 2000; Jetsche A. et al., 2006; Szyf M, et al., 1989; Keshhet I, et al., 1985).

In this respect, the idea was developed of being able to reprogram any completely differentiated cell by partially or completely modifying the methylation state of its genome, by means of treatment with specific factors.

Taranger and collaborators (Taranger C K, et al., 2005) have in fact shown that epithelial cells 293T can de-differentiate until markers are expressed typical of the embryonic state after 1 hour treatment with the extract of NCCIT cells, isolated from teratoma. Quantitative gene expression analysis and microarray study effected on the colonies maintained in a culture for 23 passages revealed that the transition to the pluripotent cell phenotype involves a dynamic stimulation of hundreds of genes typical of NCCIT cells (among which OCT4 (Ovitt C E, et al., 1998), SOX2 (M. V. Zappone, et al., 2000), NANOG (K. Mitsui, et al., 2003)) and an expression drop in genes characterizing cells 293T and generic differentiation indicators such as, for example, type A laminins.

Furthermore, experimental evidence has shown that a demethylating agent, 5′ Aza 2′ cytidine (5-AzaC), a product analogous to cytosine, can cause an extensive demethylation of the residues of 5-methylcytosine (M. Vlahovic, et al., 1999) and reduce the methyltransferase DNA activity (Tsuji-Takayama K, et al., 2004; Karpf A R, et al., 1999; U. Aapola, et al., 2001; X. Cheng, 1995; L. R. Silverman, et al., 1993; Simonsson, S., and Gurdon, 2004; Flasza, M., et al., 2003).

4 enzymes of DNA cytosine methyltransferase, Dnmt1, Dnmt3a, Dnmt3b and Dnmt31 have been identified in embryonic cells (Shiels P G, et al., 1999; Eyestone W H, et al., 1999; Wakayama T, et al., 2001).

Dnmt1 is the main enzyme which maintains the methylation state during the DNA replication. Its inactivation in murine models resulted in the loss of genomic imprinting and led to the precocious mortality of the embryo. Dnmt3a, Dnmt3b and Dnmt31, which mainly catalyze the methylation de novo, are extremely active in embryonic stem cells and promote their differentiation in vitro.

Used as an effective chemotherapeutic agent in the treatment of leukemia (Yang A S, et al., 2006), 5-AzaC has proved to be a useful experimental means for studying DNA methylation in cell differentiation and in gene activation mechanisms (J. G. Herman, et al., 1994; K. Yoshiura, et al., 1995; Y. L. Ottaviano, et al., 1994; C. M. Bender, et al., 1998). 5-AzaC, for example, has proved to intervene in differentiation induction in a myogenic sense of multipotent stem cells isolated from the umbilical cord, bone marrow and adipose tissue (Lin Y, et al., 2006). Other studies also provide data relating to its modulating activity on embryonic development with respect to proliferogenic and differentiative effects (Tsuji-Takayama K, et al., 2004). Furthermore, embryoid bodies differentiated in vitro and treated for 6 hours with 5 AzaC (1 μM) re-established the colony growth process typical of stem cells and the sensitivity to LEUKEMIA INHIBITORY FACTOR (LIF).

The demethylating action of 5-Azacitydine, with a consequent restoration of the cell multipotentiality, is widely demonstrated and verified also in plants, monocotyledons (wheat) and dicotyledons (tomatoes), in WO2005003344.

Morphological/functional analysis and gene expression study have lead to the conclusion that 5-AzaC reverses the differentiated state of embryonic cells, organized in embroid bodies, to that which can be phenotypically attributed to undifferentiated pluripotent stem cells expressing typical markers such as SSEA-1, alkaline phosphatase, Oct4, Nanog and SOX2.

Numerous other studies have described reactivation in somatic cells by treatment with 5-AzaC of silenced genes such as VHL, E-cadherin, estrogen recepter and p16. Genes of the STAT family, which have an important role in maintaining the undifferentiated state of embryonic stem cells (ES) and their capacity of self-replication by transduction of the LIF signal, are also probably the target of demethylating agents. Karpf and collaborators (Karpf A R., et al., 1999) have observed that silenced STAT genes in tumor cells of the colon are reactivated by treatment with 5-AzaC, accompanied by the reestablishment of the cell sensitivity with alpha interferon (INF-α).

It would therefore seem that treatment with 5′ Aza 2′ cytidine can have an effect on the proliferation, differentiation or de-differentiation of different cell types, belonging to animal or vegetable organisms and at various development phases (embryonic, adult, etc.).

In the light of what is specified above, there is evidently the necessity of availing of a method for the production of multipotent stem cells starting from an inexhaustible autologous cell source, easily available and free of ethical problems. It is also desirable that the method be easily controllable, repeatable and above all, that it does not lead to the neoplastic transformation of the same cells.

The authors of the present invention have now found a method for the production of multipotent stem cells which satisfies all the demands described above and which overcomes the disadvantages of the methods already known in the state of the art. The authors have in fact found advantageous alternative sources of highly differentiated somatic cells destined for generative medicine, which, subjected to demethylating treatment at suitable concentrations, represent an inexhaustible source of easily available multipotent autologous stem cells. The reprogramming of this somatic cell source represents an important solution to the problem of the scarcity of bone marrow donors and has no problems of an ethical nature.

An object of the present invention therefore relates to a method for the production of multipotent stem cells starting from highly differentiated adult somatic cells of mammals or their precursors comprising the demethylating treatment phase of said cells with 5′ Aza 2′ cytidine at a concentration ranging from 0.1 μM to 175 μM until the appearance of markers typical of the undifferentiated and multipotent embryonic state and/or the reduction in the expression of the late differentiative phase markers.

According to a preferred embodiment of the method according to the invention, when said method is carried out starting from precursors of adults somatic cells of mammals, it comprises the following phases:

a) induction of the differentiation of said precursors in mature cells by:
i) culture of the precursors of said cells until the maximum confluence is reached;
ii) treatment with differentiating factors;
iii) culture in maintenance medium free of differentiative induction agents until the expression of late differentiative phase markers is verified;
b) demethylating treatment of the highly differentiated mature cells with 5′ Aza 2′ cytidine at a concentration ranging from 0.1 μM to 175 μM until the appearance of markers typical of the undifferentiated embryonic state and/or the reduction in the expression of the late differentiative phase markers.

According to preferred embodiments of the invention, the highly differentiated adult somatic cells of mammals or their precursors are selected from adipocytes, chondrocytes and cells of smooth and skeleton musculature.

In a particularly preferred embodiment of the invention, said highly differentiated adult somatic cells of mammals or their precursors are adipocytes or precursors of adipocytes and said late differentiative phase markers are adipogenic markers, preferably selected from leptin and GLUT-4 or a combination thereof.

According to a preferred embodiment of the method according to the invention, when said method is carried out starting from adipocyte precursors, it comprises the following phases:

a) induction of the differentiation of said precursors in mature adipocytes by:
i) culture of the adipocyte precursors until the maximum confluence is reached;
ii) treatment with differentiating factors, preferably selected from insulin, isobutylmethylxanthine, dexamethasone, indomethacin or a combination thereof;
iii) culture in a maintenance medium free of adipogenic induction agents until the expression of leptin or GLUT-4 is verified;
b) demethylating treatment of the highly differentiated mature adipose cells with 5′ Aza 2′ cytidine at a concentration ranging from 0.1 μM to 175 μM until the appearance of markers typical of the undifferentiated embryonic state and/or the reduction in the expression of the late differentiative phase adipogenic markers, preferably selected from leptin and GLUT-4 or a combination thereof. Said markers typical of the undifferentiated embryonic state are preferably selected from OCT4, SOX2, NANOG, SSEA-1 and alkaline phosphates or a combination thereof.

The concentration of use of 5′ Aza 2′ cytidine is preferably selected from 100 μM, 125 μM, 150 μM and 175 μM.

In a preferred embodiment, said culture in a maintenance medium free of adipogenic induction agents of phase iii) has a duration of 7-10 days.

According to a preferred embodiment, the method of the invention also comprises a differentiation induction phase in an osteogenic sense (100 nM dexamethasone, 10 mM β-glycerophosphate, 0.05 mM ascorbic acid-2-phosphate), chondrogenic (2.0×10⁻⁴ M ascorbic acid-2-phosphate, 1 ng/ml TGFβ1) or myogenic (20% Fetal Bovine Serum (FBS), 10% Horse Serum, 1% chicken embryo extract).

The present invention relates to the use of the autologous cells obtained with the method for the preparation of a medicament for the transplant and/or cell substitution following lesion, pathology or physiological aging.

According to a further embodiment of the invention reference is made to a pharmaceutical composition comprising the autologous cells obtained with the method as defined above, as active principle, together with one or more physiologically acceptable adjuvants and/or excipients.

A further object of the invention therefore relates to a kit for the induction of multipotent staminality starting from adipocytes of mammals, said kit comprising the following components:

a) 5′ Aza 2′ cytidine;
b) markers typical of the undifferentiated embryonic state selected from OCT4, SOX2, NANOG, SSEA-1 and alkaline phosphatase.

According to an alternative embodiment of the present invention, when the starting cell source are precursors of mammal adipocytes, the multipotent staminality induction kit, comprises the following components:

a) 5′ Aza 2′ cytidine;
b) markers typical of the undifferentiated embryonic state selected from OCT4, SOX2, NANOG, SSEA-1 and alkaline phosphatase;
c) differentiating factors selected from insulin, isobutylmethylxanthine, dexamethasone and indomethacin.

Finally, the invention relates to the use of 5′ Aza 2′ cytidine at a concentration ranging from 0.1 μM to 175 μM for the induction of multipotent staminality starting from highly differentiated adult mammal somatic cells or precursors thereof. According to preferred embodiments of the invention, the highly differentiated adult mammal somatic cells or precursors thereof used can be selected from adipocytes, chondrocytes and smooth or skeleton musculature cells. The concentration of 5′ Aza 2′ cytidine is preferably selected from 100 μM, 125 μM, 150 μM and 175 μM.

The present invention is now described for illustrative but non-limiting purposes, according to its preferred embodiments, with particular reference to the figures of the enclosed drawings, in which:

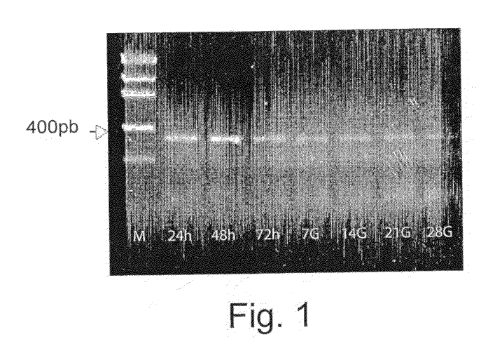

FIG. 1 shows the result of a semiquantitative PCR relating to the expression of the Ob (leptin) gene of populations of adipocytes treated with 175 μM 5-Azacytidine;

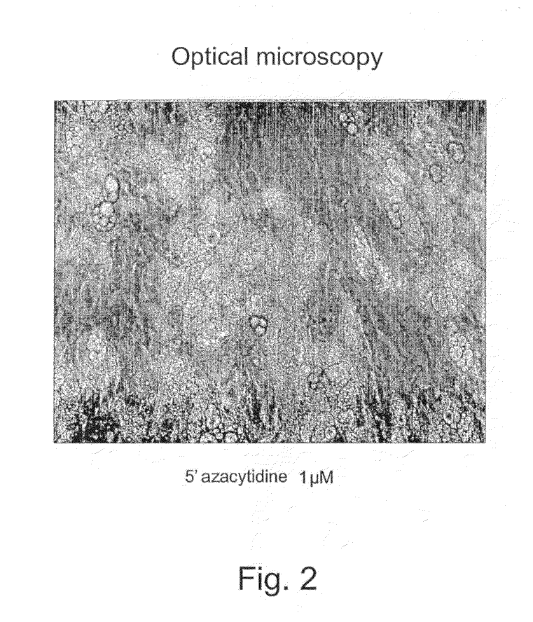

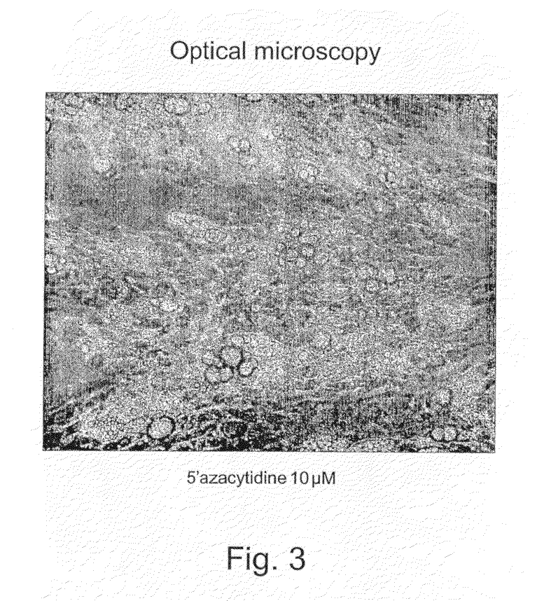

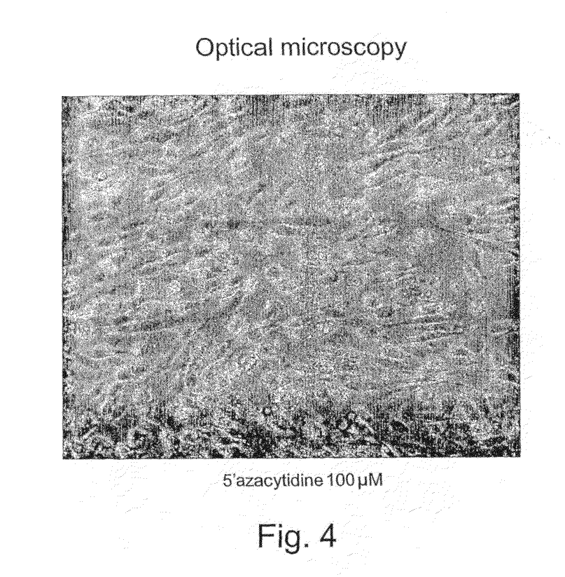

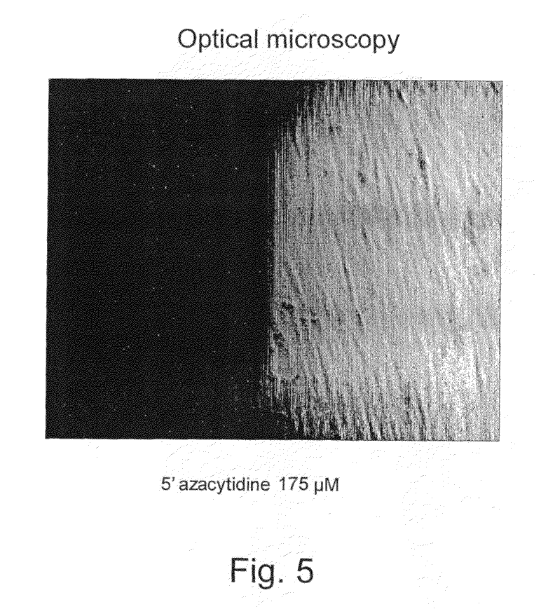

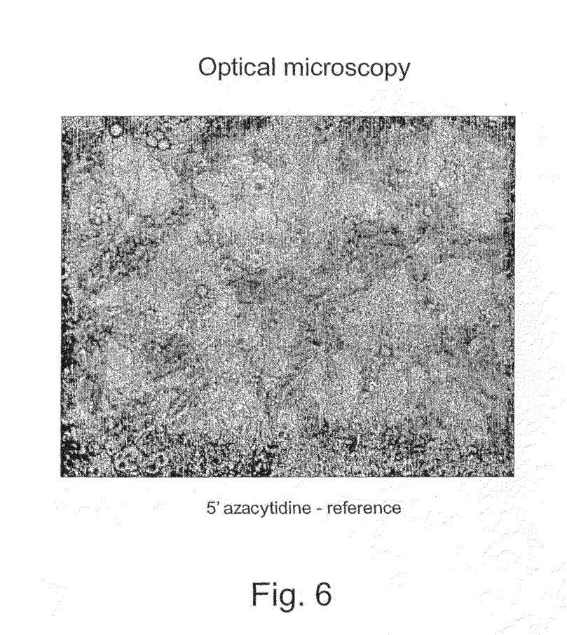

FIGS. 2-6 show the phase contrast microscope images (100× enlargement) of the cell populations after 10 days of treatment with 5-Azacytidine (1 μM, 10 μM, 100 μM, 175 μM) and mature reference adipocytes, respectively;

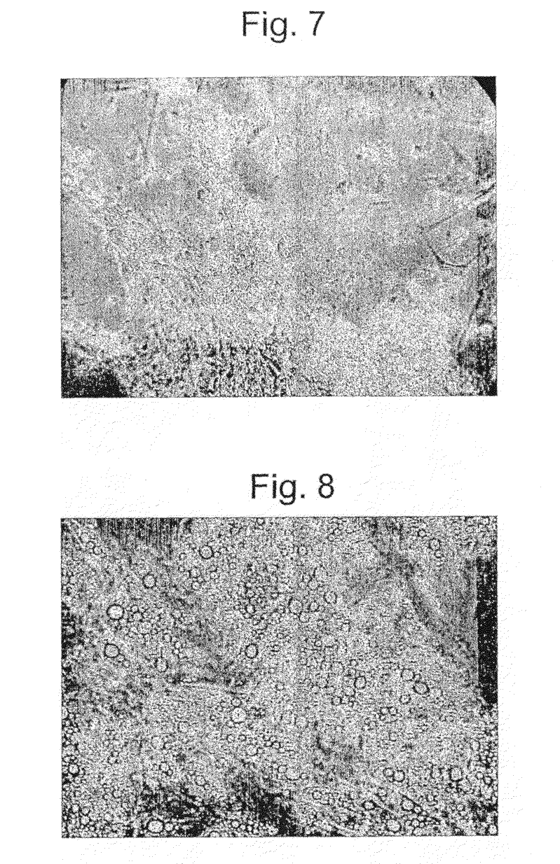

FIGS. 7 and 8 show the phase contrast microscope images (100× enlargement) of cultures treated with 5-Azacytidine at concentrations equal to 250 μM and lower than 0.1 μM, respectively.

### EXAMPLE 1

The effects were evaluated of the demethylating agent 5-Azacytidine (at different concentrations) on the capacity of mature adipose cells of reverting to the undifferentiated state and being reprogrammed, after adequate stimulation, in a multilinear differentiative sense (for example, in an osteogenic, chondrogenic and myogenic sense).

### Materials and Methods

### Development of an Adequate Cell Model In Vitro

Human pre-adipocytes and embryonic cell lines 3T3-L1 were sown at a density of 5×10³ cells/cm² and cultivated in Petri plates in a proliferative medium (DMEM-F12, 10% Fetal Calf Serum (FCS), 1% antibiotic-fungizone (AF)) until the maximum cell confluence is reached. The differentiative induction is obtained by 48 hour treatment in specific medium consisting of DMEM, 10% Fetal Bovine Serum (FBS), 1 AF and specific differentiating factors (insulin, isobutylmethylxanthine, dexamethasone, indomethacin). The mature differentiated form (univacuolar phenotype) is reached after culture of 7-10 days in maintenance medium, free of adipogenic inductive agents. Immunofluorescence studies verify the expression of leptin, a hormone typically expressed by adipocytes in adult phase.

### 5-AzaC Treatment

The mature adipose cells were maintained in a culture with DMEM, 10% FBS, 1 AF for two weeks, under continuous stimulation of 5′ Azacytidine at molar concentrations of 0.1 μM, 1 μM, 10 M, 100 μM, 125 μM, 150 μM, 175 μM, 250 μM. The optimum dose of 5′ Azacytidine ranges from 0.1 μM to 175 μM and the concentrations which are particularly effective are those between 100 μM and 175 μM. At doses ≧250 μM the demethylating treatment is no longer suitable for the production of multipotent cells starting from highly differentiated cells as it becomes a chemotherapeutic treatment which induces the cells to apoptosis rather than reverting to the undifferentiated state as shown in FIG. 7 where a high apoptotic degree is observed (treatment of cultures with 5′ Azacytidine at a concentration of 250 μM). At doses ≦0.1 μM as shown in FIG. 8, there are no signs of cell damage and/or cell proliferation in the cells treated.

### Expression of Leptin: PCR Reaction Experimental Conditions

In order to amplify the cDNA obtained from the 5-Aza cultures treated, the following were used:
Eppendorf thermocycler Amplifier;
Specific primers designed with the help of the PrimerQuest program (http://biotools.idt.com) and Nucleotide database consultation (http://www.ncbi.nlm.nih.gov) and BLAST (Basic Local Alignment Search Tool) (http://www.ncbi.nlm.nih.gov/BLAST/).

The primers were purchased from New England Biolabs.

Amplification conditions: 95° C. for 10 minutes (initial denaturation), 30 cycles of 95° C. for 30 seconds (denaturation), 52° C. for 45 seconds (annealing), 72° C. for 45 seconds (extension), 72° C. for 10 minutes (final extension). The reaction mixture was prepared with 1 μl of cDNA, 1 μl of dNtPs Mix (10 mM), 1.25 μl of MgCl₂ solution (25 mM), 1.25 μl of buffer 10×, 0.2 μl of Ampli Taq Gold (5U μl) (Applied Biosystem), 1 μl of specific sense primer and 1 μl of antisense primer (10 μM), 8 μl of H₂O RNase free in a final volume of 12.5 μl.

### Results

### Morphological Analysis of the Cultures Treated and Control by Means of Optical Microscopy

In cells treated with the demethylating agent 5-Azac, the preliminary results show a change in the morphology of the plate; it is mainly observed in fact at concentrations of 1-10 μM and higher than 100 μM, which, with the progressive disappearance of globose cells, grouped in large clusters and characterized by a voluminous lipid vacuole, is associated with the appearance of cell elements having a more elongated form with or without lipid vacuoles distributed at a cytoplasmatic level. Optic microscope images are provided hereunder of cell populations after 10 days of treatment with 5-AzaC. Phase contrast microscope images (100× enlargement) are enclosed of reference populations and those treated with 5-Azacytidine.

### Cell Growth Determination

Cell proliferation is observed by measuring the 5′-bromo-2′ deoxyuridine (BrdU) incorporated in the adipose cells treated with 5-Azacytidine. As it is known in literature that mature adipose cells do not have a proliferating capacity, it is interesting to better characterize the cell fraction having a fibroblastoid morphology which only appears in the samples treated and not in the reference samples.

### Determination of Cell Apoptosis

With the use of the TACS Klenow In Situ Apoptotic Detection kit, apoptotic phenomena were observed in both the samples treated and in the reference samples 24 hours, 48 hours, 72 hours after treatment with 5-Azacytidine. The following table indicates the results relating to the samples of adipocytes treated with 125 μM, 150 μM, 175 μM.

TABLE 1

| | | TREATED | TREATED | TREATED |
|---|---|---|---|---|
| TIME | REFERENCE | 125 μM | 150 μM | 175 μM |
| 25 hours | 10% | 15% | 22% | 38% |
| 48 hours | 12% | 15% | 28% | 41% |
| 72 hours | 10% | 10% | 10% | 10% |

### Semiquantitative (PCR) and Quantitative (RT-PCR) Tests

The expression of the late-phase adipogenic markers (leptin, GLUT-4), precocious-phase markers (PPAR-γ, CEBP-α) and markers typical of the undifferentiated embryonic state (OCT4, SOX2, NANOG, SSEA-1, alkaline phosphatase) was evaluated by means of PCR and RT-PCR.

The preliminary results show a reduction in the expression of the differentiative late-phase adipogenic markers. FIG. 1 shows the result of the semiquantitative PCR relating to the expression of the Ob gene (leptin) of adipocyte populations treated with 175 μM 5-Azacytidine.

### BIBLIOGRAPHY

Conrad C, Huss R. J Surg Res. 2005 April; 124(2):201-8.
Kiatpongsan S, Tannirandorn Y. Virutamasen P. J Med Assoc That. 2006 January; 89(1):111-7.
Saulnier N, Di Campli C, Zocco M A, Di Gioacchino G, Novi M, Gasbarrini A. Eur Rev Med Pharmacol Sci. 2005 November-December; 9(6):315-24.
Cyranoski D. Nature. 2006 Mar. 30; 440(7084):586-7.
Janes S M, Lowell S, Hutter C. J. Pathol. 2002 July; 197(4):479-91.
Strem B M, Hicok K C, Zhu M, Wulur I, Alfonso Z, Schreiber R E, Fraser J K, Hedrick M H. Keio J. Med. 2005 September; 54(3):132-41.
F. Santos, B. Hendrich, W. Reik, W. Dean. Dev. Biol. 2002; 241:172-182.
A. J. Peter, T. Daiya. Science 2001; 293:1068-1070.
R. Wolf, W. Dean, J. Walter. Science 2001; 293:1089-1093.
J. C. Gutierrez, S. Callejas, S. Borniquel, A. Martin-Gonzalez. Int. Microbiol. 2000; 3:139-146.
T. H. Bestor. Hum. Mol. Genet. 2000; 9:2395-2402.
C. Stewart, H. Stuhlmann, D. Jahner, R. Jaenisch. Proc. Natl. Acad. Sci. USA 1982; 79:4098-4102.
R. D. Palmiter, H. Y. Chen, R. L. Brinster. Cell 1982; 29:701-710.
D. Biniszkiewicz, J. Gribnau, B. Ramsahoye, F. Gaudet, K. Eggan, D. Humpherys, M. A. Mastrangelo, Z. Jun, J. Walter, R. Jaenisch. Mol. Cell. Biol. 2002; 22:2124-2135.
M. F. Chan, R. van Amerongen, T. Nijjar, E. Cuppen, P. A. Jones, P. W. Laird. Mol. Cell. Biol. 2001; 21:7587-7600.
M. Okano, D. W. Bell, D. A. Haber, E. Li. Cell 1999; 99:247-257.
Shiels P G, Kind A J, Campbell K H. Wilmut I, Waddington D, Colman A, Schnieke A E. Cloning. 1999; 1(2):119-25.
Eyestone W H, Campbell K H. J Reprod Fertil Suppl. 1999; 54:489-97.
Wakayama T, Yanagimachi R. Mol Reprod Dev. 2001 April; 58(4):376-83.
T. Haaf, M. Schmid. Cytogenet. Cell Genet. 2000; 91:113-123.
F. Santos, B. Hendrich, W. Reik, W. Dean. Dev. Biol. 2002; 241:172-182.
A. J. Peter, T. Daiya. Science 2001; 293:1068-1070.
R. Wolf, W. Dean, J. Walter. Science 2001; 293:1089-1093.
J. C. Gutierrez, S. Callejas, S. Borniquel, A. Martin-Gonzalez. Int. Microbiol. 2000; 3:139-146.
Jetsch A. Curr Top Microbiol Immunol. 2006; 301:203-25.
Szyf M, Schimmer B P, Seidman J G. Proc Natl Acad Sci USA. 1989 September; 86(18):6853-7.
Keshhet I, Yisraeli J, Cedar H. Proc Natl Acad Sci USA. 1985 May; 82(9):2560-4.
Taranger C K, Noer A, Sorensen A L, Hakelien A M, Boquest A C, Collas P. Mol Biol Cell. 2005; 16(12):5719-35.
Ovitt C E, Scholer H R. Mol Hum Reprod. 1998 November; 4(11):1021-31.
M. V. Zappone, R. Galli, R. Catena, N. Meani, S. De Biasi, E. Mattei, C. Tiveron, A. L. Vescovi, R. Lovell-Badge, S. Ottolenghi. Development 2000; 127:2367-2382.
K. Mitsui, Y. Tokuzawa, H. Itoh, K. Segawa, M. Murakami, K. Takahashi, M. Maruyama, M. Maeda, S. Yamanaka. Cell 2003; 113:631-642.
M. Vlahovic, F. Bulic-Jakus, G. Juric-Lekic, A. Fusis, S. Maric, D. Serman. Int. J. Dev. Biol. 1999; 43:843-846.
Tsuji-Takayama K, Inoue T, Ijiri Y, Otani T, Motoda R, Nakamura S, Orita K. Biochem Biophys Res Commun. 2004 Oct. 8; 323(1):86-90.
Karpf A R, Peterson P W, Rawlins J T, Dalley B K, Yang Q. Albertsen H, Jones D A. Proc Natl Acad Sci USA. 1999 November; 96(24):14007-12.
U. Aapola, R. Lyle, K. Krohn, S. E. Antonarakis, P. Peterson. Cytogenet. Cell Genet. 2001; 92:122-126.
X. Cheng. Curr. Opin. Struct. Biol. 1995; 5:4-10.
L. R. Silverman, J. F. Holland, R. S. Weinberg, B. P. Alter, R. B. Davis, R. R. Ellison, E. P. Demakos, C. J. Cornell, R. W. Carey, C. Schiffer, E. Frei III, O. R. McIntypr. Leukemia (Baltimore) 1993; 7:21-29.
Simonsson, S., and Gurdon. J. Nat. Cell Biol. 2004; 6:984-990.
Flasza, M., Shering, A. F., Smith, K., Andrews, P. W., Talley, P., and Johnson, P. A. Cloning Stem Cells 2003; 5:339-354.
Yang A S, Doshi K D, Choi S W, Mason J B, Mannari R K, Gharybian V, Luna R, Rashid A, Shen L, Estecio M R, Kantarjian H M, Garcia-Manero G, Issa J P. Cancer Res. 2006 May; 66(10):5495-503.
J. G. Herman, F. Latif, Y. Weng, M. I. Lerman, B. Zbar, S. Liu, D. Samid, D. S. Duan, J. R. Gnarra, W. M. Linehan, S. B. Baylin. Proc. Natl. Acad. Sci. USA 1994; 919:700-9704.
K. Yoshiura, Y. Kanai, A. Ochiari, Y. Shimoyama, T. Sugimura, S. Hirohashi. Proc. Natl. Acad. Sci. USA 1995; 92:7416-7419.
Y. L. Ottaviano, J. P. Issa, F. F. Parl, H. S. Smith, S. B. Baylin, N. E. Davidson. Cancer Res. 1994; 54:2552-2555.
C. M. Bender, M. M. Pao, P. A. Jones. Cancer Res. 1998; 58:95-101.
Lin Y, Liu L, Li Z, Qiao J, Wu L, Tang W, Zheng X, Chen X, Yan Z, Tian W. Mol Cell Biochem. 2006 May 23.
Tsuji-Takayama K, Inoue T, Ijiri Y, Otani T. Motoda R, Nakamura S, Orita K. Biochem Biophys Res Commun. 2004 Oct. 8; 323(1):86-90.
Cellini Francesco, Cifarelli RosaAnna Gallitelli Maria; Mango Teresa; Lauria Giuseppe; Semeraro Lucia. METHOD FOR THE ISOLATION OF EXPRESSED SEQUENCE TAGS IN PLANTS 13.01.2005. WO2005003344

1. A method for the production of multipotent stem cells starting from adipocytes or precursors of adipocytes comprising the demethylating treatment phase of said cells with 5′ Aza 2′ cytidine at a concentration ranging from 0.1 μM to 175 μM until the appearance of markers typical of the undifferentiated and multipotent embryonic state and/or the reduction in the expression of the late adipogenic differentiative phase markers.

2. (canceled)

3. The method according to any of the claims 1 wherein the concentration of 5′ Aza 2′ cytidine is selected from 100 μM, 125 μM, 150 μM, and 175 μM.

4. The method according to any claim 1 wherein said markers typical of the undifferentiated embryonic state are selected from OCT4, SOX2, NANOG, SSEA-I and alkaline phosphatase or as combination thereof.

5. The method according to claim 1, wherein said adipogenic late differentiative phase markers are selected from leptin and GLUT-4 or a combination thereof.

6. The method according to claim 1, wherein said differentiating factors of phase ii) are selected from insulin, isobutylmethylxanthine, dexamethasone, indomethacin or a combination thereof.

7. The method according claim 1 wherein said culture of phase iii) has a duration of 7-10 days.

8. The method according to claim 1, further comprising a differentiation induction phase in an osteogenic, chrondrogenic or myogenic phase.

9. use of the autologous cells obtained with the method according to claim 1, for the preparation of a medicament for the transplant and/or substitution of cells following lesion, pathology or physiological aging.

10. A pharmaceutical composition comprising the autologous cells obtained with the method according to claim 1, as active principle, together with one or more adjuvants and/or physiologically acceptable excipients.

11. A kit for the induction of multipotent staminality starting from mature mammal adipocytes, said kit comprising the following components:
a) 5′ Aza 2′ cytidine;
b) antibodies or primers for revealing markers typical of the undifferentiated embryonic state selected from OCT4, SOX2, NANOG, SSEA-1 and alkaline phosphatase and/or adipogenic late phase markers leptin or GLUT-4.

12. A kit for the induction of multipotent staminality starting from precursors of mammal adipocytes, said kit comprising the following components:
a) 5′ Aza 2′ cytidine;
b) antibodies or primers for revealing markers typical of the undifferentiated embryonic state selected from OCT4, SOX2, NANOG, SSEA-1 and alkaline phosphatase and/or adipogenic precocious phase markers PPAR-γ or CEBP-α and or adipogenic late phase markers leptin or GLUT-4;
c) differentiating factors selected from insulin. isobutylmethylxanthine, dexamethasone, indomethacin.

13. Use of 5′ Aza 2′ cytidine at a concentration ranging from for the induction of multipotent staminality starting from adipocytes or precursors thereof.

14. Use according to claim 13, wherein the concentration of 5′ Aza 2′ cytidine is selected from 100 μM, 125 μM, 150 μM, 175 μM.
